(19) United States
(12) Patent Application Publication
(10) Pub. No.: US 2004/0009197 A1
(43) Pub. Date: Jan. 15, 2004

(54) Use of resveratrol as sunscreen

(76) Inventors: Mario DeRosa, Naples (IT); Mose Rossi, Naples (IT)

Correspondence Address: Karen A Lowney, Estee Lauder, 125 Pinelawn Road, Melville, NY, 11747, US

(21) Appl. No.: 10/296,725

(22) PCT Filed: May 29, 2001
(86) PCT No.: PCT/EP01/06103

(30) Foreign Application Priority Data
Jun. 2, 2000 (IT) NA2000A000037

(51) Int. Cl. A61K 7/00

(52) U.S. Cl. 424/401

(57) ABSTRACT

Use of trans and cis resveratrol and their ether, ester, ethoxylated, glycosylated and hydroxylated derivatives as sunscreens for protection against light having a wavelength of from 200 to 320 nm.

### FIELD OF THE INVENTION

The present invention relates to the use of resveratrol and the derivatives thereof as active principles for sunscreens.

### BACKGROUND OF THE INVENTION

There is an increasing demand and need for new sunscreens which, while permitting skin tanning, help in preventing sunburns and skin diseases caused by the UV stress.

Extensive studies have been made on the ultraviolet radiations of sunlight and skylight reaching the surface of the earth and on the effects of such radiations on the human skin. Ultraviolet energy absorbed by the human skin can produce an erythema reaction (redness), whose intensity is dependent upon the amount of energy absorbed. It has been established that the radiations between 290 and 315 nm, named UV-B, are responsible of erythema and of a substantial portion of energy, which produces a retarded or indirect tanning. This is originated by the activation, between 48-72 hours, of a massive synthesis of melanin in melanocytes and by an increase of melanosomes in all the stratifications of cheratinocytes of malpighian. However the ultraviolet radiations having wavelength between 315 and 400 nm, named UV-A, promote a fast but labile tanning, which involves only the mature melanosomes and not the melanocytes of the basal zone. Ultraviolet radiation emits different quantities of energy and therefore produces an erythema reaction at different time intervals after exposure. The minimal amount of UV associated energy required to produce a perceptible redness reaction of the skin is termed “Minimal Erythema Dose” or MED.

The tanning ability is genetically predetermined and is related to the capacity to produce the melanin pigment, within the pigment cells, when stimulated by UV-B and UV-A. The extent of any erythemal response is a function of the skin color and thus less time is required to produce a MED in light skinned than in dark skinned individuals. The most rapid way to cause tanning, is to allow the sun to produce erythema of the skin. The erythema sufficient to induce a tanning, yet not so severe as to cause pain, requires only half the time of the exposure necessary to produce a painful sunburn. Sun tanning can occur at the UV-A wavelengths but it slowly develops under natural conditions. Tanning, most commonly, develops after the exposure to the UV-B band with sunburn.

During the past forty years, a great number of chemical compounds have been screened for their filtering effects in the UV range and utilized in cosmetic formulations for reducing the absorbed UV dose while modulating the erythema and tanning processes. The goal is to obtain a good tanning with the minimal injury to the exposed skin.

Whether or not a substance absorbs light in the ultraviolet range and is also a usable sunscreen for the human skin depends on several factors. In addition to the high filtering effectiveness in the erythemal range (UV-B range), it should also be compatible with the skin and the mucous membrane and must be not toxic. Finally the substance should be chemically stable and neither be altered nor discolored by ultraviolet radiation. A preparation containing the substance should be stable during storage, have no intrinsic odor, and be compatible with the commonly used cosmetic ingredients.

Sunscreen preparations which extend the time necessary for the sun to produce a sunburn are commercially available. Such preparations contain sunscreens, which are, almost exclusively, synthetic compounds, that absorb ultraviolet light at various wavelengths.

UV-A radiation causes tanning, but is weak in causing reddening of the skin. About 20-50 joules/cm² of UV-A energy is required to produce one MED. The erythema reaction is maximal in intensity about 24 hours after exposure. Suitably UV-A absorbing agents include 2,4-dihydroxybenzophenone (Uvinul 400); 2-hydroxy-4-methoxybenzophenone (oxybenzone, Spectra-Sorb UV9, Uvinul M-40); 2,2′,4,4′-tetrahydroxybenzophenone (Uvinul D50); 2,2′-dihydroxy-4,4′-dimethoxybenzophenone (Uvinul D49); 2-ethylhexyl-2-cyano-3,3′-diphenylacrylate (Uvinul N539); 2-ethylhexyl-4-phenyl-benzophenone carbonate (Eusolex 3573); 2-hydroxy-4-methoxy-4′-methylbenzophenone (mexenone, Uvistat 2211); 2-(2′-hydroxy-5′-t-octylphenyl)benzotriazole (Spectra-Sorb UV 5411); 2,2′-dihydroxy-4-methoxybenzophenone (dioxybenzone, Spectra-Sorb UV24); 2-hydroxy-4-(n-octyloxy)benzophenone (octabenzone, SpectraSorb UV531); 4-phenylbenzophenone (Eusolex 3490); and 2-(2′-hydroxy-5′-methylphenyl)benzotiazole (Tinuvin P). The UV-A absorbing compounds are present in the final product in concentration from about 0.5% to about 10% by weight of the formulation. The amount will vary according to the particular agent selected and whether the formulation is intended to minimize or permit tanning. The preferred UV-A absorbing agent is 2-hydroxy-4-methoxybenzophenone alone or in combination with 2,2′-dihydroxy-4-methoxybenzophenone.

UV-B radiation causes the sunburn reaction that also stimulates pigmentation (tanning) of the skin. Approximately 0.02-0.05 joules/cm² of UV-B energy is required to produce one MED. The erythema reaction is maximal in intensity at about 6-20 hours after exposure. Suitable UV-B absorbing agents include 4-(dimethylamino)benzoic acid ethyl ester; and isopropyl p-aminobenzoate; 4-(dimethylamino)benzoic acid-2-ethylhexyl ester (Escalol 507); 4-(dimethylamino)benzoic acid pentyl ester (Escalol 506); glyceryl p-aminobenzoate (Escalol 106) and isobutyl p-aminobenzoate (Cycloform). The UV-B absorbing agent/s are present in the final product in concentration from 1% to 15% by weight of the formulation. The amount will vary according to the particular agent selected and the degree of protection desired in the final product. The preferred UV-B absorbing agent is 4-(dimethylamino)benzoic acid, 2-ethylhexyl ester.

For human application, ultraviolet UV-B and UV-A screens are incorporated in various cosmetic oil carriers, oily solutions, oil lotions, and creams. Additionally, compounds such as hydroxyaldehydes, in particular dihydroxyacetone, imidazole and various imidazole derivatives such as 4-(hydroxymethylimidazole), may be incorporated in the formulation to provide an artificial tanning with ultraviolet protection, i.e. pigmentation of the skin which resembles natural melanin pigmentation in appearance only.

Up to now ultraviolet UV-B and UV-A screens are of synthetic origin and have had only the physical role of preventing the skin damages of UV exposure.

The present invention provides multifunctional sunscreens, that conjugate an efficient and selective filtering of UV-B radiation with specific biological actions in preventing the skin damages associated to the UV exposure, comprising as active ingredients resveratrol and the ether, ester, ethoxylated, glycosylated and hydroxylated derivatives thereof.

More particularly, the present invention relates to compositions for the topical application, containing cis or trans resveratrol or derivatives thereof, of formula (I)

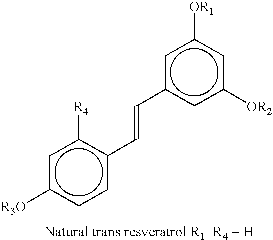

wherein:

R₁, R₂, R₃ are H; C₁-C₃₆ alkyl groups, optionally substituted by OH groups and optionally comprising one or more double bonds; C₂-C₃₆ acyl groups, optionally substituted by OH groups and optionally comprising one or more double bonds; a —(CH₂-CH₂—O)ₙ—H group where n is an integer from 1 to 30; or a glycosydic residue; and R₄ is H or OH.

Preferred resveratrol derivatives according to the invention are ethers, esters, ethoxylated, hydroxylated and glycosylated derivatives.

Particularly preferred resveratrol ether derivatives have formula (I), wherein at least one of R₁, R₂, R₃ is a C₁-C₃₆ alkyl group, optionally substituted by OH groups and optionally comprising one or more double bonds, and the others can be H; and R₄ is H.

Particularly preferred resveratrol ester derivatives have formula (I), wherein at least one of R₁, R₂, R₃ is a C₁-C₃₆ acyl group, optionally substituted by OH groups and optionally comprising one or more double bonds, and the others can be H; and R₄ is H.

Particularly preferred resveratrol ethoxylated derivatives have formula (I), wherein at least one of R₁, R₂, R₃ is a —CH₂-CH₂—O)ₙ—H group where n is an integer from 1 to 30, and the others can be H; and R₄ is H.

Particularly preferred resveratrol glycosylated derivatives have formula (I), wherein at least one of R₁, R₂, R₃ is a glycosydic residue, and the others can be H; and R₄ is H.

Particularly preferred resveratrol hydroxylated derivatives have formula (I) wherein R₁, R₂, and R₃ are H and R₄ is OH.

Resveratrol (3,4,5-trihydroxystilbene) is a phenolic stilbene and the parent glycosydes are called polydatin or piceid. The trans isomer occurs in a narrow range of spermatophytes, including principally vines, peanuts and pine trees. Resveratrol is classified as a phytoalexin and its synthesis in plants is induced by stress, in particular UV-irradiation. Resveratrol is also a potent anti-oxidant, in vivo preventing free radical propagation.

The high resveratrol content in the rhizomes of the plant Poligonum cuspidatum, makes this compound now easily available.

In vivo and in vitro experiments have shown that resveratrol possesses many biological attributes: a) is a potent anti-oxidant and a vasorelaxing compound and exerts a cardiovascular protection (The Lancet, 341:1103-1104, 1993; Neuroreport, 8:1499-1502, 1997; Chim Pharm Bull, 12:128-129, 1996; Arch Pharm Res, 13:132-135, 1990; Thrombosis and Gaemostasis, 76:818-819, 1996); b) has an anti-inflammatory action, inhibiting lypoxygenase and cyclooxygenase (Science, 267:1782-1788, 1995); c) acts as an antimutagen, by inhibiting the cellular events associated with tumor initiation, promotion and progression (Chem Pharm Bull, 30:1766-70, 1982; Science, 267:1782-1788, 1995; Am J Enol Vitic, 46:159-165, 1996; Science, 275:218-220, 1997; Cancer Res, 54:5848-5855, 1994; Anticancer Res, 14:1775-1778, 1995; Anal Biochem, 169:328-336, 1988; Proc Natl Acad Sci USA, 91:3147-3150, 1994; Proc Natl Acad Sci USA, 72:1848-1851, 1975; Carcinogenesis, 8:541-545, 1987).

None of the recent patents on the use of resveratrol in pharmaceutical and cosmetic applications (WO9959561; WO9958119; EP0773020; FR2766176; WO9904747) relate to the field of this invention.

Resveratrol UV spectrum shows absorption peaks at 216 nm (ε_{M} 19,836 and ε/g/L 87), 305 nm (ε_{M} 28,044 and ε/g/L 123) and 309 nm (ε_{M} 27,816 and ε/g/L 122), that does not depend on the solvent nature and pH values. Considering that an ideal UV-B sunscreen should have a selective absorption capacity of the solar radiation between 290 and 315 nm, the UV spectrum and the ε values of resveratrol make this molecule the most efficient compound now available as sunscreen. In fact the effectiveness of a sunscreen agent can be determined by dividing the adsorbance at the maximum peak between 290 and 315 nm (UV-B sunscreen) by the concentration in g./L. This is known as the “K” value of a sunscreen agent. The K value of resveratrol is 123 in the region of the UV-B, a value that represent a real advantage compared with the sunscreens conventionally used. In fact, the higher the K value, the better the sunscreening ability and the lower the amount of material needed for protection from sun radiation causing erythema. In other words, from the K value the amount of sunscreening agent necessary for protection from the sun ultraviolet radiation can be determined and used in any cosmetically acceptable base preparation.

The following Table reports the UV data obtained with conventional UV sunscreen, in comparison with resveratrol.

| | | Max | | | K | Solvent | |
|---|---|---|---|---|---|---|---|
| Compound | | ads. | ε_{M}* | Mw | value | System | use |
| 3,4,5-trihydroxystilbene | Resveratrol | 216 | 19,836 | 228 | 87 | Ethanol | UVB |
| | | 305 | 28,044 | | 123 | | |
| | | 309 | 27,816 | | 122 | | |
| | | 216 | 19,820 | 228 | 87 | Ph 4; 0,1 M | UVB |
| | | 305 | 28,015 | | 123 | Acetate | |
| | | 309 | 27,910 | | 122 | buffer | |
| | | 216 | 19,800 | 228 | 87 | PH 7; 0,1 M | UVB |
| | | 305 | 28,104 | | 123 | Phosphate | |
| | | 309 | 27,899 | | 122 | Buffer | |
| 4-amino benzoic acid | PABA | 283 | 15,300 | 137 | 112 | Ethanol | UVB |
| 3,3,5-trimethyl cyclohexyl- | Homosalate | 306 | 4,300 | 262 | 16 | Ethanol | UVB |
| 2-h drox benzoate | | | | | | | |
| 2-hydroxy-4- | Benzophenone-3 | 288 | 14,000 | 228 | 63 | Ethanol | UVB |
| methoxyphenylmethanone | | 329 | 9,400 | | 41 | | |
| 2-phenyl-benzimidazole-5- | Novantisol | 305 | 28,250 | 274 | 101 | 0,1 N | UVB |
| sulphonic acid | | | | | | NaOH | |
| Ethyl-4-bis(2-hydroxy- | Ethyl dihydroxy- | 311 | 27,000 | 273 | 119 | Ethanol | UVB |
| propyl)amino benzoic acid | propyl PABA | | | | | | |
| 1,2,3-propanetriol, | Glyceryl PABA | 297 | 18,700 | 211 | 89 | Ethanol | UVB |
| 2-ethylbexyl p- | Octyl dimethyl | 311 | 27,300 | 277 | 107 | Ethanol | UVB |
| dimethylaminobenzoate | PABA | | | | | | |
| 2-ethylhexyl salicylate | Octyl salicylate | 307 | 4,900 | 250 | 22 | Ethanol | UVB |
| Diethanolamine methoxy | DEA methoxy- | 285 | 24,930 | 283 | 79 | Ethanol | UVB |
| hydroxycinnamate | cinnamate | | | | | | |
| Triethanolamine salicylate | TEA salicilate | 298 | 3,000 | 287 | 10 | Ethanol | UVB |
| 2-ethylhexyl-p- | Octyl methoxy- | 311 | 23,300 | 290 | 81 | Ethanol | UVB |
| methoxycinnamate | cinnamate | | | | | | |
| 2-hydroxy-4-methoxy | Benzophenone-4 | 287 | 13,400 | 308 | 47 | Ethanol | UVB |
| benzophenone-5-sulfonic | | 326 | 8,400 | | 30 | | |
| acid | | | | | | | |
| 3-(4-methylbenzylidene)- | 3-(4-methyl- | 300 | 24,500 | 254 | 97 | Ethanol | UVA |
| bornan-2-one | benzylidene)- | | | | | | |
| | camphor | | | | | | |
| 1-p-cumenyl-3- | 4-isopropyl | 345 | 28,200 | 266 | 106 | Ethanol | |
| phenylpropane-1,3-dione | dibenzoyl | | | | | | |
| | methane | | | | | | |
| 4-t-butyl-4′- | Butyl methoxy | 358 | 34,720 | 310 | 111 | Ethanol | UVA |
| methoxydibenzoyl methane | dibenzoyl | | | | | | |
| | methane | | | | | | |

*Molar extinction coefficient

Since it is well known that the exposure to sun radiation causes skin aging and can have, in special cases, a mutagenic action, the following biological properties of the resveratrol are particularly advantageous: a) the potent anti-oxidant activity of the molecule, that prevents the propagation of radicals originated by the UV radiation on the skin; b) the anti-inflammatory action of this molecule; c) the anti-aging action on the skin related to radical protection and vasorelaxing activity of the resveratrol; d) the anti-mutagen action, characterized by the specific capacity of the resveratrol to inhibit the cellular events associated with tumor promotion, initiation and progression.

The compositions of the invention may be formulated, for example, in the form of spray, solution, oil, cream, lotion, gel and the like, together with conventional solid, semi-solid, or liquid carriers, or dilution agents, mixtures thereof, and other cosmetic auxiliaries, and optionally in association with other sunscreens and active principles.

The cosmetic treatment consists of topical applications of the resveratrol based formulation in form of spray, solution, oil, cream, lotion or gel, also in association with other sunscreens and active principles.

Resveratrol may also be used in combination with other conventional sunscreens. According to this invention, cosmetic preparations or formulations generally contain from 0.1% to 20% (w/w) of resveratrol or ethers, esters, ethoxylated, hydroxylated and glycosylated derivatives thereof. Percentages of 1% to 8% by weight represent particularly preferred ranges.

A significant improvement of resveratrol-based, moisture resistant sunscreen formulations, can be obtained by using ether and ester derivatives of resveratrol with long chain alcohols and carboxylic acids, respectively. On the contrary, to obtain formulations that have high water solubility, to allow the users to completely remove the product from their bodies and clothes with ease, ethoxylated and glycosylated resveratrol derivatives can be used.

The present invention also relates to cosmetic formulations containing resveratrol and chemical skin tanning agents, including but not limited, to hydroxyaldehydes, in particular dihydroxyacetone, imidazole and various imidazole derivatives such as 4-(hydroxymethylimidazole).

No local and/or systemic side effects have been observed during and after the application of the formulations of the invention. In addition to the physical role of UV-B sun filter, resveratrol prevents the UV-induced accelerated aging and exerts an anti-inflammatory action in the erythema response.

Resveratrol, used as a sunscreen agent, offers the following advantages compared with conventional sunscreens of the prior art:

a) resveratrol UV spectrum, with maximum absorption at 305 nm (ε_{M} 28,044) and 309 nm (ε_{M} 27,816), makes this compound an ideal highly selective UV-B sunscreen;

b) resveratrol has better sunscreen ability, in comparison with the conventionally used sunscreens (K value 123 in the UV-B region), that reduces the amount of material needed for protection from erythemal rays of the sun;

c) resveratrol UV spectrum and K values are independent on solvent and pH values, and for this reason the use of the molecule as sunscreen is compatible with a large number of cosmetic formulations;

d) resveratrol is a natural, stable compound, that can be extracted in large amount at prices compatible with the industrial use as sunscreen, from the roots of the plant Polygonum cuspidatum;

e) the potent anti-oxidant activity of resveratrol prevents the propagation in the skin of radicals originated by the UV radiation;

f) resveratrol has anti-aging action on the skin stressed by sun radiation for the coupled effects of the radical protection and the vasorelaxing activity;

g) resveratrol anti-inflammatory action limits the severe consequences of the erythema formation after exposure to the sunburn UV-B band and makes it possible a rapid and efficient tanning process of the skin;

h) resveratrol anti-mutation action, characterized by the specific capacity to inhibit the cellular events associated with tumor initiation and promotion, prevents the mutagenic action that may be due to overexposure to sun radiation;

i) resveratrol is easily adsorbed on the skin surface, originating a stable long lasting protection from the UV-B radiation;

j) resveratrol is easily soluble in the conventional components used in the sunscreen formulations, making possible high concentration of the product;

k) the lipophilic (ethers and esters with long-chain alcohols and carboxylic acids) and hydrophilic (ethoxylated and glycosylated) resveratrol derivatives provide sunscreen preparations with optimal properties of moisture resistance and water solubility, respectively.

The following Examples further illustrate the invention.

### EXAMPLE 1

Sun-Care Cream SPF 15

Formulation (concentration in % w/w): A. deionized water 69,75, polysorbate 20 2.50; disodium EDTA 0.05; xantan gum 0.20; resveratrol 5.00; glycerin 5.00; butylene glycol 4.00; B. light mineral oil 5.00; sorbitan palmitate 3.00; cetearyl octanoate 2.00; dimethicone 0.50; cocoa butter 0.80; C. bisabolol 1.00; imidazolidinyl urea 0.50; phenoxyethanol and methylparaben and ethylparaben and propylparaben and butylparaben (Phenonip, Nipa) 0.50; fragrance 0.20.

Procedure: combine A and B in separate vessel; heat at 75° C. and mix until dissolution; add B to A at 75° C.; add C to AB at 45° C.; mix 20 min until smooth and lustrous.

### EXAMPLE 2

Waterproof Sunscreen Lotion SPF 5

Formulation (concentration in % w/w): A. deionized water 87,00; hydroxypropyl methylcellulose 0.10; disodium EDTA 0.05; B. C₁₂₋₁₅ alcohols benzoate 8.00; trioleilresveratrol 4.00; C. acrylates/C₁₀₋₃₀ alkyl acrylates crosspolymer 0.25; carbomer 0.20; D. PEG-20 almond glycerides 0.20%; fragrance 0.20.

Procedure: mix A until homogeneous; combine B in a separate vessel; heat and mix until dissolution; disperse C in B; mix until well dispersed; with moderate agitation, add BC to A; mix 30 min; add D and mix until smooth and lustrous.

### EXAMPLE 3

Nongreasy Oil, SPF 10

Formulation (concentration in % w/w): cyclomethicone pentamer 71,32; dioctyl sebacate 15; phenyltrimethicone 4.00; dimethicone 0.65 cs 2; resveratrol monohexanoyl ether 6.00; octyl salicylate 3.00; methylparaben 0.01; propylparaben 0.01; butylparaben 0.01.

Procedure: Mix the Components Until Homogeneity

In the following, the results of the pharmacological tests carried out on resveratrol are reported.

1—Resveratrol as Free Radical Scavenger

The scavenging activity of resveratrol on reactive oxygen species has been studied with ESR spectroscopy. HO° radicals, generated by ultrasound irradiation (15 min, 23 kHz) of deionized water, were detected with ESR, using 10 mM DMPO as the spin-trapping agent. The HO° trapping capacity of resveratrol is evaluated over the concentration range 0-6 μM. The IC₅₀ (concentration needed for 50% inactivation process of free HO° radicals) is about 30 pM.

2—Resveratrol as Sunscreen

SPF values of the sunscreen preparations reported in the Examples 1-3, were determined with a SPF in vivo test, on female Hartley albino guinea pigs weighing about 400 g. The animals were shaved with a commercial cream 24 h before the irradiation. The test materials were applied 30 min before irradiation on the distal zone to the head, on a 5 cm² area at a dose of about 2 mg/cm², the proximal zone of the back of the head of the animals served as control site. A bank of four lamps providing a mean irradiation of about 1.2 mW cm² at 310 nm has been used as UV-B source. Following light exposures, sites were occluded with a cotton pad. SPF values were calculated as the ratio of MED of protected skin to the MED of unprotected skin. The erythema was evaluated according to the following scale: not irradiated, 0, pale pink; slight erythema, 1, pink; moderated erythema, 2, strong pink; severe erythema, 3, strong pink, edema; ulcerated erythema, 4, strong pink, ulceration. One MED (erythema grade 1) for untreated animals correspond to 5 min exposure at 400 mJ/cm². In these conditions the erythema has been observed four hours after irradiation. The SPF values of the tested resveratrol based products are the following: sun care cream of Example 1: SPF 15; waterproof sunscreen lotion of Example 2: SPF 5; nongreasy oil, of Example 3: SPF 10.

1. Use as sunscreen agents of trans and cis resveratrol or ether, ester, ethoxylated, glycosylated and hydroxylated derivatives thereof of formula (I) 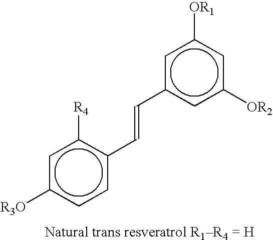
wherein:
R₁, R₂, R₃ are H; C₁-C₃₆ alkyl groups, optionally substituted by OH groups and optionally comprising one or more double bonds; C₂-C₃₆ acyl groups, optionally substituted by OH groups and optionally comprising one or more double bonds; a —(CH₂-CH₂—O)ₙ—H group where n is an integer from 1 to 30; or a glycosydic residue; and R₄ is H or OH.

2. Use as claimed in claim 1, wherein the resveratrol ether derivatives have formula (I), wherein at least one of R₁, R₂, R₃ is a C₁-C₃₆ alkyl group, optionally substituted by OH groups and optionally comprising one or more double bonds, and the others can be H; and R₄ is H.

3. Use as claimed in claim 1, wherein the resveratrol ester derivatives have formula (I), wherein at least one of R₁, R₂, R₃ is a C₁-C₃₆ acyl group, optionally substituted by OH groups and optionally comprising one or more double bonds, and the others can be H; and R₄ is H.

4. Use as claimed in claim 1, wherein the resveratrol ethoxylated derivatives have formula (I), wherein at least one of R₁, R₂, R₃ is a —(CH₂-CH₂—O)ₙ—H group where n is an integer from 1 to 30, and the others can be H; and R₄ is H.

5. Use as claimed in claim 1, wherein resveratrol glycosylated derivatives have formula (I), wherein at least one of R₁, R₂, R₃ is a glycosydic residue, and the others can be H; and R₄ is H.

6. Use as claimed in claim 1, wherein resveratrol hydroxylated derivatives have formula (I) wherein R₁, R₂, and R₃ are H and R₄ is OH.

7. Sunscreen compositions comprising resveratrol or ether, ester, ethoxylated, glycosylated and hydroxylated derivatives thereof together with a cosmetically acceptable carrier.

8. Sunscreen compositions as claimed in claim 7, containing 0.1 to 20% w/w resveratrol or derivatives thereof, preferably 1 to 8% w/w.

9. Sunscreen compositions as claimed in claims 7-8, further containing coal tar, pyrition and its derivatives, undecylenic acid and its derivatives and anti-fungine and anti-inflammatory compounds.

10. Sunscreen compositions as claimed in claims 7-9, further containing conventional sunscreens.
